(19) United States
(12) Patent Application Publication
(10) Pub. No.: US 2006/0251731 A1
(43) Pub. Date: Nov. 9, 2006

(54) Therapeutic herbal lozenge composition

(76) Inventor: Artista Marchioni, Rolling Hills Estates, CA (US)

Correspondence Address: KARAMBELAS & ASSOCIATES, 655 DEEP VALLEY DRIVE, SUITE 303, ROLLING HILLS ESTATES, CA, 90274, US

(21) Appl. No.: 11/487,800

(22) Filed: Jul. 17, 2006

Related U.S. Application Data
(63) Continuation-in-part of application No. 10/652,051, filed on Aug. 30, 2003, now Pat. No. 7,087,250.

(51) Int. Cl. A61K 33/38; A61K 36/48

(52) U.S. Cl. 424/618; 424/757; 424/773; 424/774

(57) ABSTRACT

A herbal therapeutic composition in lozenge form is provided which enhances respiration comprising colloidal silver, osha root and lobelia herb. Other herbal substituents which further enhance this novel composition are disclosed including licorice root, yerba santa leaf, eyebright herb, cats claw, rosemary, ginger, green tea leaf, grape seed, and peppermint spirits.

### I. CROSS-REFERENCE TO RELATED APPLICATIONS

This is a continuation-in-part of U.S. patent application Ser. No. 10/652,051, filed Aug. 30, 2003, now U.S. Pat. No. ______, which is incorporated by reference.

### II. FIELD OF THE INVENTION

This invention is directed to herbal substituents that are respiratory aids and, more specifically, to those compositions that are implemented in lozenge form for human consumption to mitigate both bacterial and viral infections, among other respiratory ailments.

Common colds are self-limiting viral illnesses of the upper respiratory tract. Rhino-viruses, the main viruses found in common colds, cause scratchy throats, which are followed by sneezing, runny nose, nasal congestion and other familiar symptoms. No treatment has been proven to effectively reduce the duration of common colds.

Common colds result in millions of lost or impaired work and school days with billions of dollars wasted on palliatives each year. A reliable simple side-effect free and cost effective method of reducing common cold duration would be important to the public and to the economy.

Various preparations have been employed to mitigate the effects of colds and other respiratory ailments. Between 1987 and 2004, for example, reductions in duration of common colds were reported by the ingestion of zinc lozenges in five additional double-blind, placebo controlled clinical trials and no effects were reported in five others. Consequently, by 2004 these contradictory later findings resulted in loss of interest in this method of treating common colds.

Commercial preparations sold under the name of Cold-Eze are found to reduce the severity and duration of the common cold but such preparations are not found to be a cure nor do they relieve cold symptoms. These preparations generally dial down the severity of the cold to more dealable or acceptable level for the user.

Various other treatments for respiratory ailments, especially colds, include antihistamines which work by preventing histamines from attaching to a cellular receptor to avoid sneezing, coughing, runny nose and stopped up nose during colds.

Nonsteroidal anti-inflammatory drugs such as Ibuprofen and Neproxin are effective in treating inflammation, pain and fever. These compositions, known as NSAIDs, block the production of certain natural inflammatory mediators called prostaglandins. Although NSAIDs have been used to treat general symptoms of a cold, such as feverishness, chilliness, muscle ache and general not feeling well, more recently they have been employed to treat other cold symptoms, especially cough. However, the major side effect of NSAIDs is irritation of the gastrointestinal tract in some people which can lead to gastrointestinal ulceration and bleeding or prolonged bleeding and reduced kidney function.

Decongestants, such as pseudoephedrine, may be employed to open the nasal passages by shrinking blood vessels in the mucous membrane of the nose which is the primary cause of the nasal obstruction of colds. However, these decongestants may produce rapid heart rate, blood pressure elevation and nervous stimulation. In addition, when applied directly to the mucous membrane of the nose, decongestants tend to lose their effectiveness over time and result in “rebound” obstruction and mucosal damage.

Anticholinergics are compositions that block the action of the parasympathetic nervous system on mucous gland secretion thereby reducing nasal discharge exhibiting similar activity to first generation antihistamines. These compositions differ from antihistamines in that they have no activity against histamine and so are less effective in treating sneezing. However, Anticholinergics may cause difficulty in urination in men with prostate disease and worsen the eye problems of glaucoma.

Cough suppressants may be employed which are natural narcotics like codeine and synthetic narcotics like dextromethorphan (DM). These compositions act on the brain to depress the cough reflex center and have been shown to be effective in patients with chronic cough in controlled trials but there is little published information on their effectiveness in coughs associated with colds.

These compositions can produce gastrointestinal discomfort and should not be used in patients with lung diseases where may cough may be ineffective and secretions retained in the lung.

Antiseptic lozenges/anesthetic and antibiotic throat lozenges should be used with caution in patients with myasthenia gravis, users with low plasma cholinesterase concentrations or in those receiving anticholinesterases.

Various over-the-counter throat lozenges are available, for example, Chloraseptic, in addition to over-the-counter antihistamines sold under the name of Chlortrimatron (chlorpheniramine), diphenhydramine, sold under the name of Benedryl. Common side effects of these antihistamines are sleepiness and dry mouth. Loratadine, sold under the name of Claritin, may be less sedating than the older antihistamines.

With regard to prescription antihistamines, phexophenadine, known as Allegra, cetirizine, sold under the name Zyrtec, are generally considered safe for use.

Decongestants, such as pseudoephedrine, sold under the name of Sudafed and others such as Sinutabs, are not recommended for anyone who has high blood pressure or for women that are pregnant.

Zicam is a non-drip nasal gel containing zincam gluconicum to reduce both the duration and severity of the common cold. Side effects of Zicam is the subject matter of a $75 million law suit against the company for user's loss of smell when using the product. The suit settled for $12 million.

### III. PRIOR ART

In U.S. Pat. No. 4,684,528, entitled Flavor of Zinc Supplements for Oral Use, there is disclosed formulations of zinc compounds with select amino acids such as glycine (aminoacetic acid) in a base material. The base material can be a sweetening agent such as hard or soft candy base, a syrup, chewing gum, dentifrice, or the like. The advantage of such formulations is that the unpalatable and undesirable taste and aftertaste of unformulated zinc compound and of zinc compounds mixed with a base material are markedly reduced and the products may be slowly dissolved in the mouth to achieve nutritional or therapeutic results.

In U.S. Pat. No. 4,758,439, entitled Flavor of Zinc Supplements for Oral Use, there is disclosed formulations of zinc compounds with select amino acids such as glycine (aminoacetic acid) in a base material, as in the above-recited U.S. Pat. No. 4,684,528.

In U.S. Pat. No. 5,409,905, entitled Cure for Common Cold, there is disclosed a nontoxic flavor stable, pleasant tasting composition releasing zinc (or Zn²+) from compositions containing a highly ionizable zinc compound other than zinc gluconate that reduces the duration of common colds in humans.

In U.S. Pat. No. 5,512,293, entitled Oral Sustained Release Drug Delivery Device, there is disclosed a sustained release delivery device for delivering a beneficial agent into the oral cavity of a patient.

In U.S. Pat. No. 5,686,107, entitled Chewable Pharmaceutical Tablets, there are disclosed formulations of chewable pharmaceutical tablets for delivery of prescription pharmaceutical actives, non-prescription pharmaceutical actives, or over-the-counter actives comprise as an excipient an aggregate of coprocesses microcrystalline cellulose and a galaotomannan. It is disclosed that addition of these excipients imparts improved smell, taste, texture, and mouth feel to the finished product.

In U.S. Pat. No. 6,733,749, entitled Throat Lozenges for the Treatment of Hemosphilus Influenza, there is disclosed a lozenge for the treatment of Hemophilus influenzae infections of the mouth, throat and nasal passages which comprise a lytic enzyme composition specific for Hemophilus influenzae and a lozenge carrier for delivering said lytic enzyme.

### IV. OBJECTS OF THE INVENTION

Thus there is a continuing demonstrated need to provide orally administered compositions in the form of lozenges for human beings to alleviate respiratory difficulties, for example, the common cold and aid respiration.

Therefore, an object of this invention is to provide a novel, naturally occurring composition comprising herbal components in the form of a lozenge to aid respiration, devoid of the above noted deficiencies.

Still another object of this invention is to provide a novel lozenge composition which promotes respiratory efficiency.

Still another object of this invention is to provide a lozenge composition which promotes respiration and results in no deleterious side effects.

Yet still another object of this invention is to provide an improved therapeutic lozenge composition which promotes the body's immunity to respiratory elements.

### V. SUMMARY OF THE INVENTION

These and other objects of the instant invention are accomplished generally speaking by providing a lozenge containing a therapeutic composition comprising herbal substituents and, more specifically, herbal compositions comprising colloidal silver, osha root and lobelia herb. These substituents may be employed in any suitable concentration. Typical concentrations include colloidal silver 1-3%, osha root 55-75% and lobelia 20-40%. Preferable results are obtained when these three substituents are combined in the following weight percentages: colloidal silver about 2%, osha root about 65% and lobelia about 33%. Colloidal silver is known to be an effective natural antibiotic. Although any suitable colloidal silver material may be employed, it is preferred to employ high quality colloidal silver containing small particles approximately 1000^{th} of a micron employing a true electrocolloidal process. These colloidal solutions are found to remain stable in increasing concentrations of silver. Osha root has enhanced antibacterial action and promotes proper lung and bronchial action; lobelia has been found to enhance stress reduction. The unique herbal composition as recited above is found to have far superior therapeutic effects on the entire respiratory system as opposed to the collective additive effects of each of the three components. This unique therapeutic herbal composition may be further enhanced by adding licorice root. Licorice root has been found to enhance reduction or elimination of mucous and improved adrenal action. A typical herbal composition would be employed in the following concentration ranges: colloidal silver about 1-2%, osha root about 40-50%, lobelia herb about 15-30%, licorice root about 15-30%. A preferred composition including these four substituents is colloidal silver at about 1.6%, osha root at about 50%, lobelia at about 25%, and licorice root at about 25%. It is likewise found that further enhancement may be obtained by adding yerba santa leaf which has been found to enhance the drying of mucosal tissue when secretions are excessive. Typical concentrations of this five substituent herbal therapeutic composition include colloidal silver 0.6-2%, osha root 25-45%, lobelia 10-25%, licorice root 10-25%, yerba santa leaf 15-35%. A preferred composition including these five substituents is colloidal silver at about 1.2%, osha root at about 36%, lobelia at about 18%, licorice root at about 18%, and yerba santa leaf at about 27%.

Still further enhancements may be obtained by employing additionally the eyebright herb which is known to enhance the reduction of allergic response, for example, hay fever and overactive immune systems. A typical six herbal substituent composition containing eyebright would include colloidal silver about 0.5-1.5% by weight, osha root about 20-40% by weight, lobelia about 10-20% by weight, licorice root about 10-20% by weight, yerba santa leaf about 10-30% by weight, and eyebright herb about 10-30% by weight. A preferred composition including these six substituents is colloidal silver at about 0.95%, osha root at about 28%, lobelia at about 14%, licorice root at about 14%, yerba santa leaf at about 21%, and eyebright herb at about 21%.

In addition, it is found that further enhancements and therapeutic effects to the core three component herbal composition may be realized by further adding cats claw bark which is known to enhance stress reduction and inflammation, rosemary leaf which is a known antioxidant and increases circulation, and ginger root which enhances anti-inflammatory action and improves circulation, in addition to grape seed which provides enhanced antioxidant action in addition to enhanced immune response. A typical seven substituent composition would be employed in the following concentration ranges: colloidal silver at about 0.5-1.5%, osha root at about 20-30%, lobelia at about 5-20%, licorice root at about 5-20%, yerba santa leaf at about 10-30%, eyebright herb at about 10-30%, and rosemary at about 2-4%. A typical nine substituent composition would be employed in the following concentration ranges: colloidal silver at about 0.4-1.2%, osha root at about 15-35%, lobelia at about 5-20%, licorice root at about 5-20%, yerba santa leaf at about 10-30%, eyebright herb at about 10-30%, rosemary at about 2-4%, ginger at about 2-4%, and further comprising cats claw at about 2-4%. A preferred ten substituent composition is colloidal silver at about 0.85%, osha root at about 25%, lobelia at about 12½%, licorice root at about 12½%, yerba santa leaf at about 19%, eyebright herb at about 19%, cats claw at about 3%, rosemary at about 3%, ginger at about 3%, and grape seed at about 1.3%.

The herbal substituents lobelia, yerba santa, licorice root and ginger root are found to perform better than their dry analogs so that if supplies are readily available it is preferred to employ the fresh version of these herbal substituents.

A highly preferable composition embodying the three substituent core composition and others as recited above could include colloidal silver at about 0.4-1.2% by weight, osha root at about 15-20% by weight, lobelia at about 5-15% by weight, licorice root at about 5-15% by weight, yerba santa leaf at about 10-20% by weight, eyebright at about 10-20% by weight, and cats claw, rosemary and ginger at about 1½-4½% by weight, with grape seed being employed at about ½-1½% by weight. A typical composition including these nine substituents is colloidal silver at about less than 1%, osha root at about 25%, lobelia at about 13%, licorice root at about 13%, yerba santa leaf at about 20%, eyebright herb at about 20%, cats claw at about 3%, rosemary at about 3%, and ginger at about 3%. A particularly preferred composition which is found to be most effective in smoggy areas is found to have the following composition: colloidal silver about 0.85%, osha root about 25%, lobelia about 12½%, licorice root about 12½%, yerba santa leaf about 19%, eyebright herb at about 19%, cats claw at about 3%, rosemary at about 3%, ginger at about 3%, and grape seed at about 1.3%. It should be noted that peppermint spirits may be added in very small amounts to provide a more pleasing taste to this unique herbal composition.

Although these herbal substituents may be combined in any suitable manner to effect respiratory relief, the following ranges of composition for each of the substituents has been found to be preferable in application: osha root 10-30%, eyebright herb 5-20%, yerba santa leaf 5-20%, licorice root 2-20%, lobelia herb 2-20%, cats claw bark 0.5-5%, rosemary leaf 0.5-5%, ginger root 0.5-5%, green tea leaf 1-10%, peppermint spirits 0.05-0.2%, grape seed 1-4% and colloidal silver 0.02-0.15%.

Various solvents may be employed to provide a solution of these herbal substituents as well as various extraction techniques to provide the preferred concentrations. Any suitable solvent may be employed in providing the herbal substituents of the instant inventions. Typical solvents include water and alcohol among others.

Any suitable method of extraction may be employed in providing the herbal substituent composition of the instant invention. Typical extractions include those using grain alcohol solvents among other alcohols and conventional liquids used in solvent extractions.

Although the herbal substituents of the therapeutic respiratory composition of the instant invention may be combined in any suitable ratio including those recited above, the following working example has been found to produce optimal results when using this composition.

A composition of 930 milligrams per milliliter is produced employing any suitable solvent extraction technique to provide osha root in a concentration of 22% by weight or approximately 205 milligrams per milliliter; eyebright herb at 16.5% by weight or approximately 153 milligrams per milliliter; yerba santa leaf at 16.5% by weight or approximately 153 milligrams per milliliter; licorice root at 11% by weight or approximately 102 milligrams per milliliter; lobelia herb at 11% by weight or approximately 102 milligrams per milliliter; cats claw bark at 2.7% by weight or approximately 25 milligrams per milliliter; rosemary leaf at 2.7% by weight or approximately 25 milligrams per milliliter; ginger root at 2.7% by weight or approximately 25 milligrams per milliliter; green tea leaf at about 4.7% by weight or approximately 44 milligrams per milliliter; peppermint spirits at about 0.12% by weight or approximately 1.1 milligrams per milliliter; grape seed and grape seed extract at 0.08% by weight or approximately 2.5 milligrams per milliliter; and colloidal silver at approximately 30 parts per million silver or about 0.00279 milligrams per milliliter.

In addition to alleviating the adverse symptoms resulting from smog, for example in Los Angeles, recently named again the smoggiest city in the United States, additional advantages may be employed for support of the respiratory system employing the liquid composition of the instant invention. This herbal composition has been found to enhance the immune system in alleviating the affects of colds, flu, allergies, asthma, bronchitis and environmental irritation to respiratory tissues.

In addition the commercial incarnation of this herbal composition referred to as SMOG CHECK®) SILVER RESERVE has been found to benefit smokers, athletes and those who suffer from altitude sickness as it enhances the ability of the lung to employ oxygen and has been found to be safe and effective for long term use.

A practical application for use of this composition is suggested during airplane flights of any duration to boost or enhance the immune system from attack by airborne irritants.

In addition to those benefits listed above, this novel composition has been found to be an aid to those with sinus congestion, lung congestion, bacterial or viral infections, stress symptoms, inflammation and circulatory problems. This therapeutic respiratory composition has been found to enhance antibacterial action, antiviral action, antiallergenic action, improve oxygenation, provide antihistamine action, act as an antioxidant, enhance stress alleviation, provide enhanced anti-inflammatory action, reduce coughing and mucous production, increase circulation and enhance antimicrobio action.

Although each of the individual substituents have shown characteristics as follows, any one of them may be substituted with a suitable substituent in a suitable concentration to provide the same properties: osha root has enhanced antibacterial action and promotes proper lung and bronchial action; eyebright enhances the reduction of allergic response in, for example, hay fever and overactive immune systems; yerba santa has been found to enhance the drying of mucosal tissue when secretions are excessive; licorice root has been found to enhance reduction or elimination of mucous and improved adrenal action; lobelia has been found to enhance stress reduction; green tea has been found to enhance antioxidants and enhance the immune system; cats claw bark has been found to enhance stress reduction and inflammation; rosemary leaf has been found to enhance antioxidant action and increase circulation; ginger root has been found to enhance anti-inflammatory action and improve circulation; peppermint has been found to enhance alleviation of sinusitis; and grape seed has provided enhanced antioxidant action in addition to enhanced immune response.

Any suitable carrier may be employed with the lozenge herbal composition of the instant invention. Typical carriers include rice syrup, citric acid, natural cherry flavor, other fruit flavors, maltitol syrup, sucrose, cornstarch, acacia, gum tragacanth, anethole, linseed, oleoresin, mineral oil, cellulose and other binders and combinations thereof. In addition, menthol from mint oil or peppermint oil may also be employed to further enhance the therapeutic benefits of these lozenges.

While the present invention has been particularly described with respect to preferred concentrations of preferred substituents, it will be understood that the invention is not limited to these particular concentrations and substituents described in the preferred embodiments. For example, the optimal combination of this therapeutic composition may be varied accordingly by substituting for the above substituents, substitute substituents in suitable concentrations that possess the same or similar properties as outlined above to obtain the same or similar results as above recited. It is intended to cover all alternatives, modifications, and equivalents as may be included within the spirit and scope of the invention defined by the appended claims.

The herbal compositions of the instant invention may be employed in varying concentrations according to the requirements of the user and the results sought to be obtained. Typically, the above recited herbal compositions are employed in concentrations of at least 5%, for example, for repeated use by children and up to 95% for repeated use by severely afflicted adults, with concentrations of 75% employed where added ingredients and excipients in increased amounts are desired to obtain specific results. It should be noted that preferably and optimally where quick and immediate response is indicated, a maximum percentage of 80-95% should be employed depending on the stability and cohesiveness of the delivery system with regard to the binders and excipients employed.

Any suitable process may be employed to provide the herbal compositions of the instant invention in lozenge form. Conventional methods include providing the herbal substituents in fine particulate form and then either encapsulating them or interspersing a binder with excipients to form a lozenge. In the alternative, if it is found preferable to work from liquid compositions which have employed conventional extraction processes, these compositions may be exposed to conventional freeze dried techniques rendered particulate and thereafter either encapsulated or blended with the above recited binders and excipients as desired to provide a lozenge.

The lozenge of the instant invention may comprise encapsulating the liquid rather than just providing particulate herbal compositions in varying degrees of herbal liquid compositions with varying viscosities. In addition, the salutary and therapeutic properties of menthol may be incorporated in the compositions of the lozenge of the instant invention, although the concentrations of menthol may be varied according to the intended use, typically about 10 milligrams of menthol may be added to each lozenge which is approximately the size of a conventionally available cough drop.

What is claimed is:

1. A therapeutic respiratory herbal composition lozenge comprising colloidal silver, osha root and lobelia in combination with a carrier.

2. The composition as defined in claim 1 wherein said colloidal silver is in a concentration of about 1-3%, osha root is in a concentration of about 55-75%, and lobelia is in a concentration of about 20-40%.

3. A therapeutic respiratory herbal composition lozenge comprising colloidal silver at about 2%, osha root at about 65%, and lobelia at about 33% in combination with a carrier.

4. The composition as defined in claim 1 further comprising licorice root.

5. The composition as defined in claim 4 wherein said colloidal silver is in a concentration of about 1-2%, osha root is in a concentration of about 40-50%, lobelia is in a concentration of about 15-30%, and licorice root is in a concentration of about 15-30%.

6. A therapeutic respiratory herbal composition lozenge comprising colloidal silver at about 1.6%, osha root at about 50%, lobelia at about 25%, and licorice root at about 25% in combination with a carrier.

7. The composition as defined in claim 4 further comprising yerba santa leaf.

8. The composition as defined in claim 7 wherein said colloidal silver is in a concentration of about 0.6-2%, osha root is in a concentration of about 25-45%, lobelia is in a concentration of about 10-25%, licorice root is in a concentration of about 10-25%, and yerba santa leaf is in a concentration of about 15-35%.

9. A therapeutic respiratory herbal composition lozenge comprising colloidal silver at about 1.2%, osha root at about 36%, lobelia at about 18%, licorice root at about 18%, and yerba santa leaf at about 27% in combination with a carrier.

10. The composition as defined in claim 7 further comprising eyebright herb.

11. The composition as defined in claim 10 wherein said colloidal silver is in a concentration of about 0.5-1.5% by weight, osha root is in a concentration of about 20-40% by weight, lobelia is in a concentration of about 10-20% by weight, licorice root is in a concentration of about 10-20% by weight, yerba santa leaf is in a concentration of about 10-30% by weight, and eyebright herb is in a concentration of about 10-30% by weight.

12. A therapeutic respiratory herbal composition lozenge comprising colloidal silver at about 0.95%, osha root at about 28%, lobelia at about 14%, licorice root at about 14%, yerba santa leaf at about 21%, and eyebright herb at about 21% in combination with a carrier.

13. The composition as defined in claim 10 further comprising rosemary.

14. The composition as defined in claim 13 wherein said colloidal silver is in a concentration of about 0.5-1.5%, osha root is in a concentration of about 20-30%, lobelia is in a concentration of about 5-20%, licorice root is in a concentration of about 5-20%, yerba santa leaf is in a concentration of about 10-30%, eyebright herb is in a concentration of about 10-30%, and rosemary is in a concentration of about 2-4%.

15. The composition as defined in claim 13 further comprising ginger.

16. The composition as defined in claim 15 wherein said colloidal silver is at about 0.4-1.2%, osha root is at about 15-35%, lobelia is at about 5-20%, licorice root is at about 5-20%, yerba santa leaf is at about 10-30%, eyebright herb is at about 10-30%, rosemary is at about 2-4%, ginger is at about 2-4%, and further comprising cats claw at about 2-4%,

17. A therapeutic respiratory herbal composition lozenge comprising colloidal silver at about less than 1%, osha root at about 25%, lobelia at about 13%, licorice root at about 13%, yerba santa leaf at about 20%, eyebright herb at about 20%, cats claw at about 3%, rosemary at about 3%, and ginger at about 3% in combination with a carrier.

18. The composition as defined in claim 15 further comprising grape seed.

19. A therapeutic respiratory herbal composition lozenge comprising colloidal silver at about 0.85%, osha root at about 25%, lobelia at about 12½%, licorice root at about 12½%, yerba santa leaf at about 19%, eyebright herb at about 19%, cats claw at about 3%, rosemary at about 3%, ginger at about 3%, and grape seed at about 1.3% in combination with a carrier.

20. The composition as defined in claim 18 further comprising green tea leaf.

21. A therapeutic respiratory herbal composition lozenge comprising osha root at about 10-30%, eyebright herb at about 5-20%, yerba santa leaf at about 5-20%, licorice root at about 2-20%, lobelia herb at about 2-20%, cats claw at about 0.5-5%, rosemary leaf at about 0.5-5%, ginger root at about 0.5-5%, green tea leaf at about 1-10%, grape seed at about 1-4%, and colloidal silver at about 0.02-0.15% in combination with a carrier.

22. The composition as defined in claim 20 further comprising peppermint spirits.

23. A therapeutic respiratory herbal composition lozenge comprising osha root at about 10-30%, eyebright herb at about 5-20%, yerba santa leaf at about 5-20%, licorice root at about 2-20%, lobelia herb at about 2-20%, cats claw at about 0.5-5%, rosemary leaf at about 0.5-5%, ginger root at about 0.5-5%, green tea leaf at about 1-10%, grape seed at about 1-4%, colloidal silver at about 0.02-0.15%, and peppermint spirits at about 0.05-0.2% in combination with a carrier.
